(19) United States
(12) Patent Application Publication
(10) Pub. No.: US 2019/0060040 A1
(43) Pub. Date: Feb. 28, 2019

(54) High Volume Dental Suction Tip with Lower Volume Suction Port

(71) Applicant: William l. Barham, Mt. Airy, NC (US)

(72) Inventor: William l. Barham, Mt. Airy, NC (US)

(21) Appl. No.: 16/042,346

(22) Filed: Jul. 23, 2018

Related U.S. Application Data
(60) Provisional application No. 62/549,031, filed on Aug. 23, 2017.

(51) Int. Cl. A61C 17/06

(52) U.S. Cl. CPC A61C 17/043; A61M 1/008

(57) ABSTRACT

A dental suction tip that can be connected through a hose to a low pressure or vacuum source will entrain material in a patient's mouth. A flap valve is formed by incisions in the hollow tube forming the suction tip. The flap valve is located between opposite ends of the dental suction tip so that the flap valve remains aligned with the tube walls so as not to restrict entraining flow in the suction tip. The dental suction tip can be reoriented with the tube extending between the patient's lips so that the patient can exert pressure to force valve flaps inwardly to open the valve to permit the patient to spit into the dental suction tip. The flap valve is spaced from the open end of the suction tip by a distance sufficient to prevent simultaneous blockage of both openings to avoid stalling or suck back or fluid migration and resulting adverse effects on patient health without compromising evacuating flow.

### CROSS REFERENCE TO PRIOR CO-PENDING APPLICATION

This application claims the benefit of prior co-pending U.S. Provisional Patent Application 62/549,031, filed Aug. 23, 2017.

### BACKGROUND OF THE INVENTION

### Field of the Invention

This invention is intended for use in suctioning fluids and debris particles from a cavity, such as from the mouth by use of a disposable dental suction tip, during a dental procedure as well as providing a secondary valve which will allow the patient to expectorate into the dental suction tip by reorientation of the dental suction tip.

### Description of the Prior Art

During active dental procedures debris would accumulate within a patient's mount unless the debris or material is removed in some manner. Suction is commonly used to remove particulate and fluid debris generated as part of the dental procedure, as well as saliva or water or fluid spray injected into the patient's mouth during the active procedure. Vacuum or suction tips, such as the prior art device shown in FIGS. 1 and 2 are commonly mounted on a hose. A pump or other means for generating a partial vacuum or air flow is connected to the vacuum or suction tip by the hose. The flow created in the vacuum tip, placed in the patient's mouth, is effective in removing most but not all of the debris created during an active dental procedure. An opening or mouth in the distal end of the suction tip can be placed in the vicinity of the oral cavity, such as adjacent to a molar that may be drilled, and the suction air flow though the vacuum tip will entrain much of the debris in the patient's mouth.

Prior art vacuum or suction tips are not ideally suited for comfortably removing all of the fluid or particulate debris or deposits that may accumulate within the patient's mouth. Typically the patient's will need to or feel a need to spit to clear his or her oral cavity, even though there may not be excess fluid or particulate debris present in the active vicinity of the dental procedure. Thus the patient may wish to spit into a spit sink or a dental assistant will need to insert a separate instrument into the patient's mouth to allow the patient to spit, even though the patient may not be expelling material that would not normally be present in the oral cavity. Of course this means that instruments may need to be removed by a dentist in order to allow a dental assistant to insert a second suction device into an oral cavity of limited volume. When the patient attempts to spit, expelled or partially expelled fluid will accumulate on the patient's lips and he or she or a dental assistant will need to wipe away such excess, and because the patient's oral cavity may be numbed, this uncomfortable or abnormal feeling will create a perceived need to spit and wipe even if significant deposits may not be present. Alternatively larger particulate matter, which cannot be removed by the conventional vacuum or suction flow through the suction tips and hose, maybe generated, and will need to be actively expelled or expectorated by the patient.

In addition to discomfort that may be felt by the patient, the need to spit or expectorate forces the dentist to pause in the active portion of the dental procedure. Therefore the time to completely perform the required dental procedure or operations will be increased. Of course the patient will wish that the entire procedure be completed within the shortest possible time. Such delays will also impair the economic efficiency for the dentist, which means that he and his associates can perform fewer operations or procedures within a given time period. The result is either higher cost for the patient or reduced revenue to the dentist or dental professional.

Unlike conventional suction or vacuum tips, the instant invention is a suction tip that can be used both to suction debris or material during active dental operations or procedures, but can also be reoriented to allow the patient to spit or expectorate through a secondary side valve or port into the dental suction tip during inactive intervals to both reduce the time for both the patient and the dental professional, but also to limit the time required to clean the patient's lips and numbed mouth after spitting. The Center for Disease Control (CDC) has expressed concern about “suck back” and/or fluid migration in prior art dental suction tips of High Vacuum Evacuator (HVE) tube because of a perceived adverse effect on patient's safety. The instant invention provides an HVE that can avoid or minimize stalling or decrease in suction flow.

### SUMMARY OF THE INVENTION

A dental suction tip according to this invention has a length sufficient for insertion into the mouth of a patient for removal of material during an active dental procedure. The dental suction tip comprises a cylindrical tube having a first end for disposal into the mouth of the patient and a second end attachable though a hose to a source of suction so that the suction acts to suck material from the mouth of the patient through the dental suction tip and the hose. A flap is formed on the cylindrical tube spaced from the first and second end. The flap is formed by an incision in the cylindrical tube to form a flap valve. The flap valve will remain in a neutral closed position during active stages of the dental procedure or operation so that the vacuum pressure is not reduced at the tip and not to restrict flow rate or prevent a flow loss through the dental suction tip and the hose. The flap can be deflected by pressure exerted by the patient to open the flap valve port when the cylindrical tube is reoriented and placed between the patient's lips. In this position, the patient can expectorate excess material, not removable by suction through the tube during active stages of the dental procedure in order, into a lower vacuum flow through the tip without pulling delicate tissues into the tip.

A dental suction tip according to another aspect of this invention can be connected to a pressure less than ambient to induce a flow in the dental suction tip for removing material from a patient's mouth during an active dental procedure. This dental suction tip comprises a hollow cylindrical tube, one end forming a first opening, which is positionable within a patient's mouth for removing material from a patient's mouth through the dental suction tip. A deformable valve is located on a side of the hollow cylindrical tube and is shiftable inwardly when subjected to an external pressure in excess of ambient pressure to form a second opening. Additional material can be laterally injected into the dental suction tip through this deformable valve at a position spaced from the forward end. This deformable valve on the side of the hollow cylindrical tube remains more aligned with the exterior of the hollow cylindrical tube when subjected only to a pressure less than ambient within the cylindrical tube so as not to diminish flow in the dental suction tip.

A dental suction tip according to a third aspect of this invention can be used to remove material from a patient's mouth during active dental operations and during intervals between active dental operations. This dental suction tip has a hollow cylindrical tube having first and second ends. The first end has a die cut S-shaped configuration forming an opening though which material is removed from a patient's mouth during active dental operations. The second end is attachable to an external low pressure so that air flows through the hollow cylindrical tube withdrawing material from the patient's mouth. A flap is formed on a side of the hollow cylindrical tube and spaced between the first and second ends of the hollow cylindrical tube. This flap is formed by intersecting die cut slits formable by a die moving in the same direction in which a die moves to form the S-shaped configuration. The flap, when in a neutral, unstressed configuration, is aligned with the hollow cylindrical tube so as not to inhibit flow through the dental suction tip during active dental operations. This flap is also deflectable inwardly to form a second opening for removal of material during intervals between active dental operations. The flap valve is spaced from an adjacent open end of the dental suction tip, to prevent simultaneous blockage of both the open end and the flap valve by the patient's mouth so that a vacuum pressure generating suction in the tip is not stalled during a dental procedure in which patient safety is not compromised.

### BRIEF DESCRIPTION OF THE FIGURES

FIG. 1 is a top view of a Prior Art dental suction tip that exhibits some of the basic features of the instant invention.

FIG. 2 is a side view of the Prior Art dental suction tip shown in FIG. 1, which employs an S-shaped distal edge or opening, which can be positioned in a patient's mount in proximity to an area in which material that may result from an active dental procedure is to be removed from the patient's mouth by suction.

FIG. 3 is a top view of one embodiment of this invention.

FIG. 4 is a side view of the dental suction tip shown in FIG. 3, showing a flap valve created by an incision of slit in the cylindrical tubular body of the dental suction tip. In FIG. 4, the flap valve is shown in the closed position that does not diminish flow rates or suction during an active dental procedure or operation.

FIG. 5 is also a view showing the same side of the dental suction tip as depicted in FIG. 4, but showing a side opening created by inward deflection of the two wings forming the flap valve. External pressure applied to the flap valve opens the flap valve so that material may be injected into the dental suction tip from the side. This opening allows the patient to spit or expectorate into the dental suction tip during intervals between active dental operations.

FIG. 6 is a side view of a dental suction tip with an alternate version of a flap valve in which longitudinal slits forming the flap valve are curved and converge towards the transverse slit at the center of the flap valve.

FIG. 7 is a side view of a dental suction tip with another alternate version of a flap valve in which longitudinal slit forming the flap valve are also curved and diverge towards the transverse slit at the center of the flap valve.

FIG. 8 is a side view of a dental suction tip with U-shaped flap valve.

FIG. 9 is a view showing the position of the dental suction tip inserted into a patient's open mouth in which an active dental procedure can be performed.

FIG. 10 is a view showing the dental suction tip of FIG. 9 after being removed and reoriented between the patient's lips so that the patient can spit into the dental suction tip during inactive intervals in a dental procedure.

FIG. 11 is a side view showing the same position as demonstrated in FIG. 10 with the dental suction tip between the patient's lips.

### DETAILED DESCRIPTION OF THE PREFERRED EMBODIMENTS

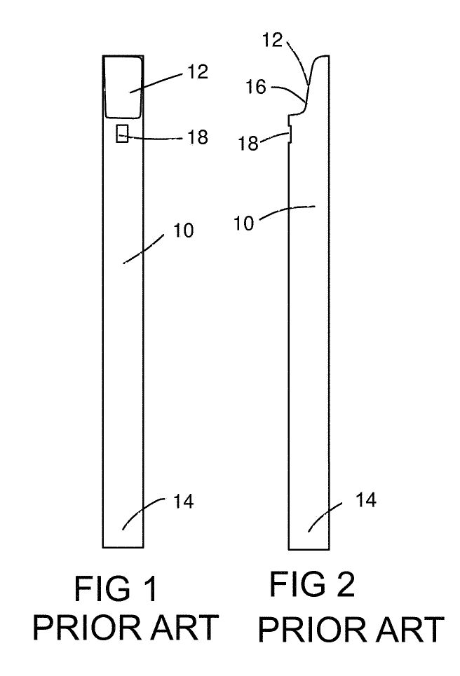

One prior art dental suction tip that includes certain basic elements also employed in the preferred embodiments of this invention is shown in FIGS. 1 and 2. This prior art dental suction tip comprises a relatively thin wall, but also relatively rigid, plastic tubular member 10, having at least an S-shaped edge 16 on the distal end 12, is employed. Other plastic tubular members can have a straight angled edge. Both the S-shaped edge and the straight angled edge could from an inclined distal end 12 on the tubular vacuum member 10. In this prior art version, a side facing opening or port 18 is also present, adjacent the distal end 12, on the tubular member shown in FIGS. 1A and 1B. This hole 18 provides additional flow for a high volume tip. It should be understood, however, that all of the various embodiments of the protective pad 20 can be employed with tubular members that do not have a hole 18 in addition to the open distal end 12. Indeed such a port need not be employed with the instant invention. Prior art versions as well as the instant invention have a second end 14 that can be attached to a flexible hose, which is in turn attached to a partial vacuum or lower pressure sink which will induce a flow in both the hose and the dental suction tip.

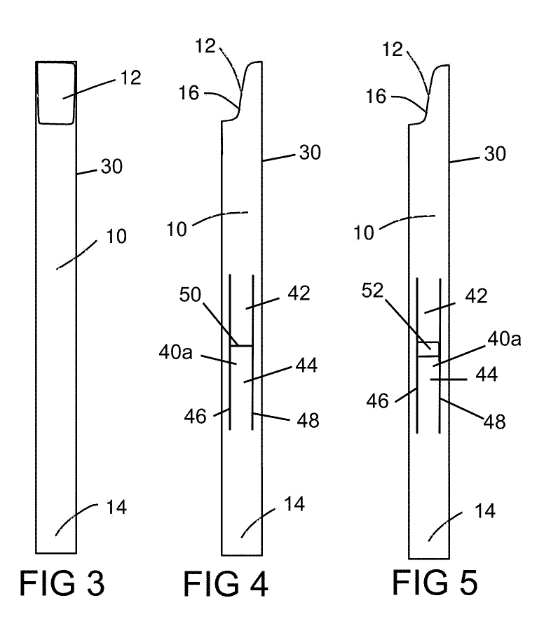

The various embodiments of a dental suction or vacuum tip 20 or high volume evacuation tip (HVE), represented in FIGS. 3-8, differ from the prior art as represented by FIGS. 1 and 2, by the valve or port 40 located between the distal end 12 and the proximate end 14. This valve or port 40 will eliminate the need for an opening 18 of the type employed in the prior art. The valve or flap valve 40 is depicted herein as multiple valve embodiments 40a-40d, the details of which will be subsequently discussed in more detail. The dental suction tip 20 of the instant invention includes a cylindrical tubular member or hollow tube 30, which can have an S-shaped edge 18 defining a mouth or scoop 16 in the distal or forward end 12. As with the conventional vacuum tip 10, this S-shaped edge or mouth 16 is to be positioned adjacent the area in which an active dental procedure is being performed. For example, if a patient's molar is being drilled, then the dental suction tip 20 will be positioned with the S-shaped mouth 16 adjacent this molar, where suction can entrain particulate matter as the molar is being drilled. During this active procedure, the valve 40 will remain aligned with adjacent portions of the wall 48 of the cylindrical tubular member 30 so that the valve will be in a closed position in which air or the flow will not bypass the full length of the dental suction tip 20. Thus the flow rate will not be diminished at the active S-shaped mouth 16, and therefore the effectiveness of the dental suction tip 20 and the entire suction or vacuum system will not be significantly reduced. With the valve 40 in this closed position, the flaps 42 and 44, which are laterally arcuate, will be generally in alignment with the tubular wall 30a in the vicinity of the valve 40. It should be understood that the alignment of the flaps 42 and 44 with the tubular wall 30a, in the closed position, need not be perfect, but any leakage through the valve 40 in the closed position should remain small given practical manufacturing considerations. For example, the valve flaps 42 and 44 are defined by longitudinal incisions, such as slits 46 and 48 in FIG. 4, and a transverse slit 50 also shown in FIG. 4. These slits will create a small gap, even in the closed position, and some leakage will occur. The flaps 42 and 44 may also be slightly deformed during fabrication and may not be perfectly aligned with the tubular wall 30a. In the closed position represented by FIG. 4, the valve flaps 46 and 48 will be significantly more closely aligned with the tubular wall 30a, then when the flap valve 40 is in the open position as represented by FIG. 5. For example, liquids and solid particulate matter will not pass in any significant volume through the flap valve 40 in the closed position. When the flap valve 40 is in the open position, as depicted in FIG. 5, liquids and solid particulate material will be injected through the opening 52 through the open valve 40 into the flow stream though the dental suction tip 20.

Although not intended to be a complete recitation of all valves that can be employed with this invention, four alternate valve configurations 40a, 40b, 40c and 40d are shown in FIG. 4-8, with FIGS. 4 and 5 showing the same embodiment in first the closed position and then in the open position.

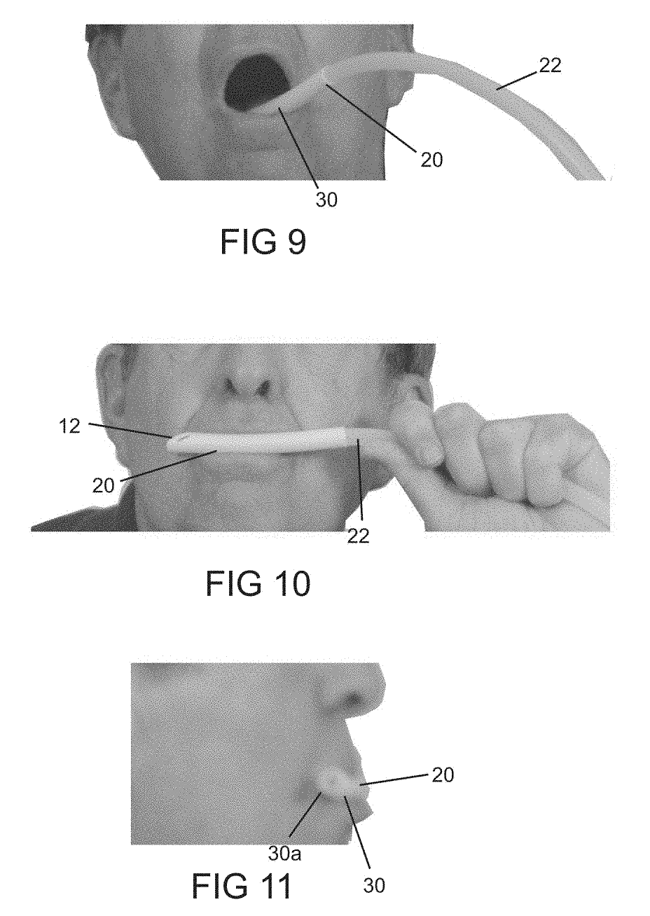

The flap valve 40 of FIGS. 4 and 5 is formed by incisions in an H-shaped configuration. Longitudinal incisions, cuts or slits 46 and 48 are parallel to each other and also parallel to an axis about which the hollow tube 30 is formed. The transverse slit 50 extends between the two longitudinal slits preferably, but not necessarily, adjacent the center of the two longitudinal slits 46 and 48. As depicted herein the transverse slit 50 is perpendicular to the two longitudinal slits or incisions 46 and 48, but perpendicularity is not essential. First and second flaps 42 and 44 are formed on opposite sides of the transverse slit 50, These flaps 42 and 44 form deflectable cantilever beams that can be inwardly deflected to leave the opening 52 between the transverse edges of each inwardly deflected flaps 42 and 44 when external pressure is applied. In a preferred embodiment of this invention the length of the flap valve 40a will be 0.75 inch (1.805) cm. inclusive of both flaps 42 and 44. The spacing of the flap valve 4a from the farthest end of the mouth 16 will be one inch (2.54 cm). These dimensions will be sufficient to prevent both the flap valve 40a and the open end 16 from being simultaneously closed to cause the vacuum to be stalled when used to evacuate fluids, debris or spit from the patient's mouth. Each flap 42 and 44 will be laterally arcuate as these flaps are formed by incisions in the cylindrical tube 30, as will be subsequently discussed in more detail. The manner in which this external pressure would be applied for any of the representative embodiments will be discussed with reference to FIGS. 9-11.

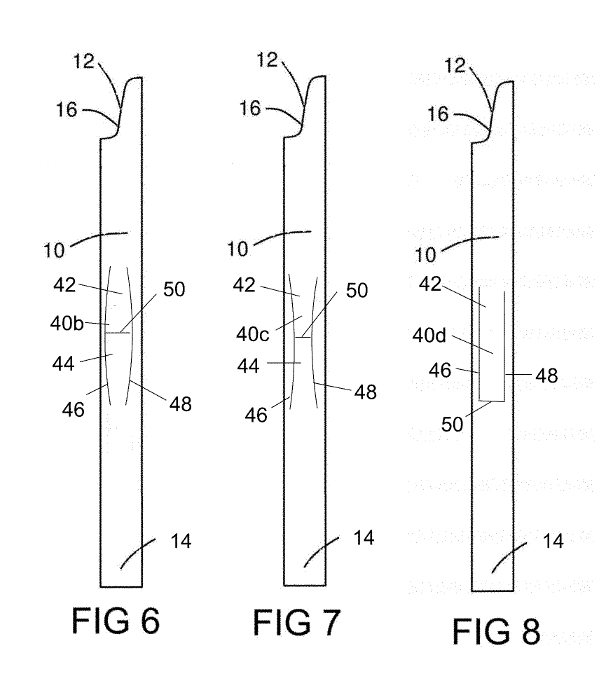

The embodiment of FIG. 6 differs from the embodiment of FIGS. 4 and 5 in that the longitudinal incisions or slits 46 and 48 are curved instead of being straight as in FIGS. 4 and 5. The incisions or slits 46 and 48 thus diverge from each other towards the center of the flap valve 40b and the transverse slot 50 in FIG. 6. There are two functional differences resulting from these curved longitudinal slits. First the width of the transverse slit or incision can be greater so that the area of the valve opening 52 in this configuration can be greater than in at least some of the other embodiments. Secondly the root of the cantilever flaps 42 and 44 in this configuration can be weaker than in other configurations. When viewed together the diverging slots 44 and 48 in FIG. 6 can result in a larger valve opening relative to the amount of external pressure applied to open the flap valve 40b. Thus the same opening can be formed with less pressure or more pressure can result in the creation of a relatively larger opening than may be possible with other configurations.

The embodiment of FIG. 7 also depicts a flap valve 40c formed with curved longitudinal incisions or slots 46 and 48 in which the longitudinal incisions converge toward the center of the flap valve 40c and the transverse slot 50. The cantilevered flaps 42 and 44 will thus be stronger than the embodiment of FIG. 6. This configuration will therefore be less likely to leak than the embodiment of FIG. 6.

A third embodiment is shown in FIG. 8. In this embodiment, the flap valve 40d is formed by a single flap 42 and the valve has a U-shaped configuration. Again these four embodiments are merely intended to represent various configurations of a dental suction tip with a flap valve, and are not intended to be exhaustive of the many configurations that could be employed.

In each of the embodiments depicted in FIGS. 3-8, the flap valves 40 are formed on the sides of the tubular member 30 and the incisions, cuts, slits or edges defining the flap valves 40 extend in the same direction as the S-shaped edges 18 at the forward end of the dental suction tip 20. This arrangement offers certain manufacturing advantages. A dental suction tip according to this invention can be fabricated using a hollow plastic tube. A plastic tube suitable for fabrication of the dental suction tip 20 can have a right circular cross section with an outer diameter of approximately 16 mm. and a wall thickness of approximately 0.50-1.0 mm. The thickness of a preferred embodiment would be 0.9 mm. Wall thicknesses within this range will permit the flaps forming the flap valve 40 to flex in response to external pressure applied by a patient when spitting into the tube through the flap valve 40 and will permit the flaps to return to their normal closed position when this external pressure is relieved. The length of the dental suction tip could be four to five cm. although different lengths could be employed. A polypropylene tube can be suitable for fabricating the dental suction tip 20, although other materials could be employed. A cylindrical tube of this size and of this type of material can be flattened during a die cut fabrication process in which the S-shaped edge 16 is cut. If the incisions to form the flap valve 40 (40a-40d) are simultaneously die cut, then these incisions 46, 48, 50 will be located on the same tube side as the S-shaped profile 16 is formed. The die cut S-shaped profile 16 can be cut between adjacent sections of extruded tubing, with the two tips 20 formed on each side of the S-shaped profile 16 being reversely oriented so that there is n material loss. Even though the two tips 20 formed in this manner are reversely oriented, the ports or flap valves 20 in each can be die cut by a common punch and the two dental suction tips 20 formed in this manner will be identical. Thus the die cut can be made in the same operation, or if the separate cuts are made in different stages, it will not be necessary to reorient the tube relative to the straight line path of the die when making these cuts or incisions. In the representative embodiments discussed herein, the incisions would not be cut on opposite sides of the tube so the travel of the die would not be sufficient to cut all the way through the flattened tube as would be necessary when fabricating the S-shaped profile 16. This reduce travel could be achieved by fabricating the die so that it would not have a cutting edge height as great as the cutting edge forming the S-shaped profile 16. To reduce material cost and fabrication time mirror images of the fabricated suction tip could be progressively formed along the length of a blank cylindrical tube. The S-shaped profile 16 would be such that only a single cut would be necessary to progressively fabricate opposed tubes with S-shaped profiles being formed on abutting tubes, with the resultant S-shaped profiles being opposed. If the flap incisions are formed only on a single side, then abutting tubes fabricated in this manner would have the flap valves 40 oriented on different sides, resulting in for example on right hand dental suction tip following one left hand dental suction tip. Alternatively the flap valve incisions could be made on two opposed sides, and sequentially fabricated tubes would be identical.

After the die cut steps are completed, the plastic tube will have sufficient memory to return to its original circular cylindrical configuration. There may be some slight offset of the valve flaps 42 and 44 from the cylindrical tubular wall 30a, but this offset will be small and the flaps can still be said to remain in alignment with the remainder of the suction tip wall. When the incisions forming the flaps 42 and 44 are completed, the neutral, unstressed position of the flaps 42 and 44 will be in the closed position shown in FIGS. 4 and 6-8. Pressure must be applied to the exterior of the flap valve 40 in order to depress the flaps 42 and 44 inwardly to open the valve. The arcuate contour of the flaps 42 and 44 will tend to retard any outward deflection of the flaps 42 and 44.

This fabrication process is not the only method by which the dental suction tips 20 could be fabricated, although it would be a simple and efficient fabrication process requiring no more time or steps than fabrication of the conventional prior art dental suction tips.

The method of employing the same dental suction tip 20 alternatively during active stages of a dental procedure and during intervals to allow the patient to spit is depicted in FIGS. 9-11. During active stages of the dental procedure, for example while the dentist is drilling the flap valve remains in a neutral, closed position to restrict flow rate reduction through the dental suction tip 20 and an attached hose 22. The position of the dental suction tip in this stage is shown in FIG. 9. The flap is deflectable by pressure exerted by the patient to more open the flap valve when the cylindrical tube is reoriented and placed between the patient's lips so that the patient can expectorate excess material, not removable by suction through the tube during active stages of the dental procedure, through the dental suction tip and into the hose. Both FIGS. 10 and 11 demonstrate the position of the dental suction tip during these intervals allowing the patient to spit.

As shown in FIG. 9, the dental suction tip 20 is place in the patient's open mouth with suction applied through the hose 22. Normally a dental assistant would hold the dental suction tip 20 in this position while the dentist performs the active dental procedure or operation, such as drilling a tooth. The S-shaped opening or mouth 16 on the forward end of the dental suction tip can be held by a dental hygienist the immediate vicinity of the active dental procedure. Suction applied in this area will entrain matter, such as debris from the active dental procedure and fluids that may accumulate. No substantial reduction of flow rate will result because the flap valve 40 is in the closed position.

When the patient feels a need to spit or actively remove material from his or her mouth, the dental hygienist can merely move the dental suction tip 20, while suction remains applied through the hose 22 to the position shown in FIGS. 10 and 11. In this position the flap valve 40 will be between the patient's lips and centrally positioned between the patient's nose and chin. The patient's lips will easily conform to the cylindrical exterior of the dental suction tip and his or her lips will easily close off the area around the flap valve 40. When the patient applies external pressure to spit, the flaps 42 and 44 will deflect inwardly forming a valve opening 52 as shown in FIG. 5. Flow through the dental suction tip 20 still continues into the hose 22, and although the forward end 12 remains open, the flow will entrain fluids and other material expectorated by the patient out of the dental suction tip 20 and through the hose 22 for subsequent discharge. The suction, along with the close fit between the patient's lips and the cylindrical tube 30 will also prevent fluid from flowing out of the mouth and along the lips, which would require the patient to wipe his or her lips, thus reducing the time need to spit and allowing the dentist to resume the active part of the dental procedure.

The flap valve 20 is normally closed and therefore differs from other dental suction tips which may employ lateral openings in a hollow tube to either prevent backflow of contaminants into the dental suction tip into the patient's mouth or to prevent soft tissue from blocking the primary intake. When soft tissue is drawn into a dental surgical tip by the vacuum or suction, this soft tissue may be damaged or injured. The flap valve of this invention can address these problems, but since the flap valve is normally closed, the flow rate through the dental suction tip is not adversely affected. Furthermore these prior art apertures do not provide an opening that will permit the discharge of significant amounts of material if the patient were to attempt to spit through such small, normally open apertures.

One of the important aspects of this dental suction tip is that this suction tip 20 will prevent stalling of the flow either when the suction tip 20 is employed to remove matter from the patient's mouth through the mouth 16 formed at the open distal end of the suction tip or through flap valve 40. Stalling occurs when flow through the suction tip 20 and through the vacuum line is interrupted when all open paths through the suction tip 20 are closed. Stalling can result in suck back and/or fluid migration in dental suction tips. If the vacuum flow is stopped or diminished due to decreasing tip vacuum, safety can be adversely affected. If the flow is stalled during a dental procedure, contaminants, which may be present in the vacuum line, may be introduced into the patient's oral cavity when vacuum flow is resumed. This can occur because spit or saliva in the tube can flow back into the patient's mount, along with contaminants which may have been present in the flow line can flow back into the patient's mount, even potentially when the patient sucks fluid back into his or her own mouth. The suction tip 20 of the instant invention incorporates no-stall protection.

In the present invention, if the entire S-shaped opening or mouth 16 is closed when the patient's lips close around the exterior of the suction tip adjacent the mouth opening 16, the secondary flap valve 40 will open inwardly since there will still be a pressure differential across the secondary flap valve 40. When the blockage of the end of the suction tip by the patient's lips, tongue or cheek is removed, the flap valve 40 will return to its essentially closed position due to the memory of the plastic ‘living hinge” at the ends of the flap valve 40. An additional outward force on the internal surfaces of the flap valve 40 generated by the air flow will also push the flap valve 40 to its neutral, closed position.

Another potential source of contamination if the flow is stalled is due to the fact that the dental suction tip, normally held by a dental technician, and not be a dentist performing a dental procedure is held at an angle of approximately forty-five degrees during normal evacuation of the oral cavity. This angle is because the dental assistant or the assistant's hands are generally held at an elevated position relative to the patient. If the vacuum is stalled, this can allow debris from the vacuum hose, which is not normally cleaned between dental procedures and is not replaced like the suction tip, to fall into the suction tip and potentially into contact with the patient's oral cavity.

The flap valve 40 is spaced from the mouth 16 or the end of the tube by a sufficient distance so the patient's mouth, lips, tongue and cheek, acting separately or together is not large enough to close both the open end 16 and the flap valve 40, always leaving one flow path open.

Since the inner diameter of the suction tip 20 is at least as great as the inner diameter of the hos with which it is used, maximum flow can be maintained through the suction tip 20 when the flap valve is closed, since the flaps will be aligned with the walls of the suction tip 20. Maximum flow is important to a dentist because the larger the suction, the larger will be the strength of the vacuum vortex formed at the end of the suction tip 20. This maximum vortex will catch or entrain a maximum amount of overspray and debris from a drilling procedure. A maximum vortex will decrease the amount of water, saliva or tooth dust that can migrate to the back of the patient's throat. Therefore, the time, which the patient will need to clear his or her throat will be minimized. The patient's comfort will also be increased because he or she will experience less discomfort due to a “coated feeling” due to matter deposited on the very sensitive soft palate and lateral walls of the throat.

The representative embodiments specifically depicted herein are not the only versions of a dental suction tip according to this invention. Modifications can be made to the structure of the deformable valve referred to herein as a flap valve. For example, different valve contours may be suitable for use by patients of different ages. Alternatively left or right handed configurations may be more suited to a specific dentist or dental hygienist. The characteristics of the flap valve can also be modified by employing extruded plastic in which the thickness of the tubing is circumferentially variable. A weaker valve could be formed in a thinner section of extruded tubing. A stronger valve would be formed in a section of the extruded tubing that is thicker than the remainder of the tubing. Other modifications may be made in order to enhance the capabilities of a dental suction tip in accordance with this invention. For example changes may be made to the flap valve contour to alter the acoustic characteristics of the dental suction tip or to cancel other noises, such as a potentially aggravating noise made by a drill. These modifications would, however, still remain within the scope of this invention as defined by the claims presented herein.

I claim:

1. A dental suction tip insertable into a patient's mouth and comprising a hollow tube connectable to low pressure sink to entrain material in the patient's mouth through a front end of the tube and discharge the material through a rear end of the tube, the dental suction tip including a flap valve formed by incisions in the hollow tube so that the flap valve can be deflected by a local external pressure applied to the flap valve by the patient to alter flow through the flap valve.

2. The dental suction tip of claim 1 wherein the flap valve is opened inwardly upon the application of an external pressure so that the patients can laterally discharge material into the dental suction tip through the flap valve for entrainment through the dental suction tip.

3. The dental suction tip of claim 2 wherein the flap valve is spaced from an adjacent open end of the dental suction tip, to prevent simultaneous blockage of both the open end and the flap valve by the patient's mouth so that a vacuum pressure generating suction in the tip is not stalled during a dental procedure in which patient safety is not compromised.

4. The dental suction tip of claim 3 wherein the open end has an S-shaped contour and the flap valve is spaced from the farthest edge of the open end by a minimum of one inch so that a patient's lips cannot simultaneously block both the open end and the flap valve.

5. The dental suction tip of claim 4 wherein the length of the flap valve, including two flaps forming the flap valve, is 0.750 inch.

6. A dental suction tip having a length sufficient for insertion into the mouth of a patient for removal of material during a dental procedure, the dental suction tip comprising:
a cylindrical tube having a first end for disposal into the mouth of the patient and a second end attachable though a hose to a source of suction so that the suction acts to suck material from the mouth of the patient through the dental suction tip and the hose;
a flap formed on the cylindrical tube spaced from the first and second end, the flap formed by an incision in the cylindrical tube so that the flap forms a flap valve, with the flap valve remaining in a neutral closed position during active stages of the dental procedure to restrict flow rate reduction through the dental suction tip and the hose, the flap being deflectable by pressure exerted by the patient to more open the flap valve when the cylindrical tube is reoriented and placed between the patient's lips so that the patient can expectorate excess material, not removable by suction through the tube during active stages of the dental procedure, through the dental suction tip and into the hose.

7. The dental suction tip of claim 6 wherein the cylindrical tube has a circular cross section and the flap valve has an arcuate cross section coincident with the circular cross section when the flap valve is in a neutral closed position.

8. The dental suction tip of claim 6 wherein the incision forming the flap valve includes a first portion extending longitudinally along the cylindrical tube and a second portion extending transversely from and relative to the first portion.

9. The dental suction tip of claim 8 wherein the first portion extends parallel to an axis of the cylindrical tube.

10. The dental suction tip of claim 8 wherein the first portion of the incision is curved as it extends longitudinally along the cylindrical tube.

11. The dental suction tip of claim 8 wherein the second portion extends perpendicular to an axis of the cylindrical tube.

12. The dental suction tip of claim 1 wherein the incision comprises an H-shaped incision.

13. The dental suction tip of claim 1 wherein the incision comprises a U-shaped incision.

14. The dental suction tip of claim 1 wherein the first end of the cylindrical tube has an S-shaped contour.

15. The dental suction tip of claim 1 wherein the incision is formed on the cylindrical tube in an orientation so that the dental suction tube must be rotated about an axis of the cylindrical tube to position the flap valve between the patient's lips upon removal of the dental suction tube from the patient's mouth.

16. A dental suction tip connectable to a pressure less than ambient to induce a flow in the dental suction tip for removing material from a patient's mouth during an active dental procedure, the dental suction tip comprising: a hollow cylindrical tube, one end forming a first opening, which is positionable within a patient's mouth for removing material from a patient's mouth through the dental suction tip, a deformable valve on a side of the hollow cylindrical tube being shiftable inwardly when subjected to an external pressure in excess of ambient pressure to form a second opening through which material can be laterally injected into the dental suction tip at a position spaced from the one end, the deformable valve on the side of the hollow cylindrical tube remaining more aligned with the exterior of the hollow cylindrical tube when subjected only to a pressure less than ambient within the cylindrical tube so as not to diminish flow in the dental suction tip.

17. The dental suction tip of claim 16 wherein the deformable portion of the cylindrical tube comprises an incision extending through one side thereof.

18. The dental suction tip of claim 17 wherein the incision extends both longitudinally and laterally relative to an axis around which the cylindrical tube is formed.

19. The dental suction tip of claim 17 wherein the incision comprises a transverse slit intersecting two longitudinally extending slits.

20. A dental suction tip for use in removing material from a patient's mouth during active dental operations and during intervals between active dental operations, the dental suction tip comprising:
a hollow cylindrical tube having first and second ends, the first end comprising a die cut S-shaped configuration forming an opening though which material is removed from a patient's mouth during active dental operations, and the second end is attachable to an external low pressure so that air flows through the hollow cylindrical tube withdrawing material from the patient's mouth;
a flap formed on a side of the hollow cylindrical tube and spaced between the first and second ends of the hollow cylindrical tube, the flap being formed by intersecting die cut slits formable by a die moving in the same direction in which a die moves to form the S-shaped configuration, and wherein the flap, when in a neutral, unstressed configuration, is aligned with the hollow cylindrical tube so as not to inhibit flow through the dental suction tip during active dental operations, and wherein the flap is deflectable inwardly to form and second opening for removal of material during intervals between active dental operations and wherein the hollow cylindrical tube is formed from a plastic which is deflectable for die cutting the S-shaped configuration and the slits forming the flap.
